(19) United States
(12) Patent Application Publication
(10) Pub. No.: US 2020/0379231 A1
(43) Pub. Date: Dec. 3, 2020

(54) OBSERVATION APPARATUS

(71) Applicant: OLYMPUS CORPORATION, Tokyo (JP)

(72) Inventor: Masahito DOHI, Tokyo (JP)

(73) Assignee: OLYMPUS CORPORATION, Tokyo (JP)

(21) Appl. No.: 16/997,156

(22) Filed: Aug. 19, 2020

Related U.S. Application Data
(63) Continuation of application No. PCT/JP2018/032376, filed on Aug. 31, 2018.

(30) Foreign Application Priority Data
Feb. 20, 2018 (JP) 2018-027604

(51) Int. Cl. G02B 21/14; G02B 21/02; G02B 5/132; G02B 21/08; C12M 1/00; C12M 1/06

(52) U.S. Cl. CPC G02B 21/14; G02B 21/02; G02B 5/132; G02B 21/36; C12M 23/14; C12M 27/02; G02B 21/086

(57) ABSTRACT

An observation apparatus includes: an illumination optical system that radiates illumination light into a container from outside the container; an objective lens that collects signal light from a cell in the container; a detection optical system that detects the signal light collected by the objective lens; and a retroreflective member that has an array of a plurality of small reflective components, is disposed across from the illumination optical system with the container interposed therebetween, and reflects the illumination light transmitted through the container.

### CROSS-REFERENCE TO RELATED APPLICATIONS

This is a continuation of International Application PCT/JP2018/032376, with an international filing date of Aug. 31, 2018, which is hereby incorporated by reference herein in its entirety.

This application is based on Japanese Patent Application No. 2018-027604, with Japanese filing date of Feb. 20, 2018, the contents of which are incorporated herein by reference.

### TECHNICAL FIELD

The present invention relates to observation apparatuses. In particular, the present invention relates to an observation apparatus for observing cells in a suspension-culture container.

### BACKGROUND ART

In the field of regenerative medicine using cultured cells, including iPS cells, in recent years, there has been a demand to scale-up the culturing process. Examples of culturing methods include the adhesion culturing method and the suspension culturing method. The adhesion culturing method involves culturing cells within a small container, such as a well plate or a dish. The suspension culturing method involves culturing cells in a floating state in a culture solution within a large container, such as a bioreactor. For producing a large number of cells, culturing methods have been changing from the adhesion culturing method to the suspension culturing method (e.g., see Japanese Unexamined Patent Application, Publication No. 2017-140006). In Japanese Unexamined Patent Application, Publication No. 2017-140006, an image of the cells in the container is acquired for ascertaining the culture status of the cells in the container.

### SUMMARY OF INVENTION

An aspect of the present invention provides an observation apparatus for observing a cell in a suspension-culture container. The observation apparatus includes: an illumination optical system that radiates illumination light into the container from outside the container; an objective lens that collects signal light from the cell in the container; a detection optical system that detects the signal light collected by the objective lens; and a retroreflective member that has an array of a plurality of small reflective components, is disposed across from the illumination optical system with the container interposed therebetween, and reflects the illumination light transmitted through the container.

### BRIEF DESCRIPTION OF DRAWINGS

FIG. 1 is an overall view of the configuration of an observation apparatus according to an embodiment of the present invention.

FIG. 2 illustrates an example of a container used in the observation apparatus in FIG. 1.

FIG. 3 illustrates a configuration example of a retroreflective member of the observation apparatus in FIG. 1.

FIG. 4 is a partial view of the configuration of a modification of the observation apparatus in FIG. 1.

FIG. 5 is a partial view of the configuration of another modification of the observation apparatus in FIG. 1.

FIG. 6 is a partial view of the configuration of another modification of the observation apparatus in FIG. 1.

FIG. 7 is a partial view of the configuration of another modification of the observation apparatus in FIG. 1.

FIG. 8 is an overall view of the configuration of another modification of the observation apparatus in FIG. 1.

FIG. 9 is an overall view of the configuration of another modification of the observation apparatus in FIG. 1.

FIG. 10 is an overall view of the configuration of another modification of the observation apparatus in FIG. 1.

FIG. 11 is an overall view of the configuration of another modification of the observation apparatus in FIG. 1.

FIG. 12 is an overall view of the configuration of another modification of the observation apparatus in FIG. 1.

FIG. 13 is an overall view of the configuration of another modification of the observation apparatus in FIG. 1.

FIG. 14 is a partial view of the configuration of another modification of the observation apparatus in FIG. 1.

FIG. 15 is an overall view of the configuration of another modification of the observation apparatus in FIG. 1.

FIG. 16A is a partial view of the configuration of another modification of the observation apparatus in FIG. 1.

FIG. 16B illustrates a light source in FIG. 16A, as viewed along an optical axis of an objective lens.

FIG. 17A illustrates the configuration of a modification of the retroreflective member.

FIG. 17B is a cross-sectional view of the retroreflective member in FIG. 17A.

FIG. 18 is a cross-sectional view of another modification of the retroreflective member.

FIG. 19A illustrates a modification of the arrangement of the retroreflective member.

FIG. 19B illustrates a specific example of the arrangement of the retroreflective member in FIG. 19A.

FIG. 19C illustrates another specific example of the arrangement of the retroreflective member in FIG. 19A.

### DESCRIPTION OF EMBODIMENTS

An observation apparatus 1 according to an embodiment of the present invention will be described below with reference to the drawings.

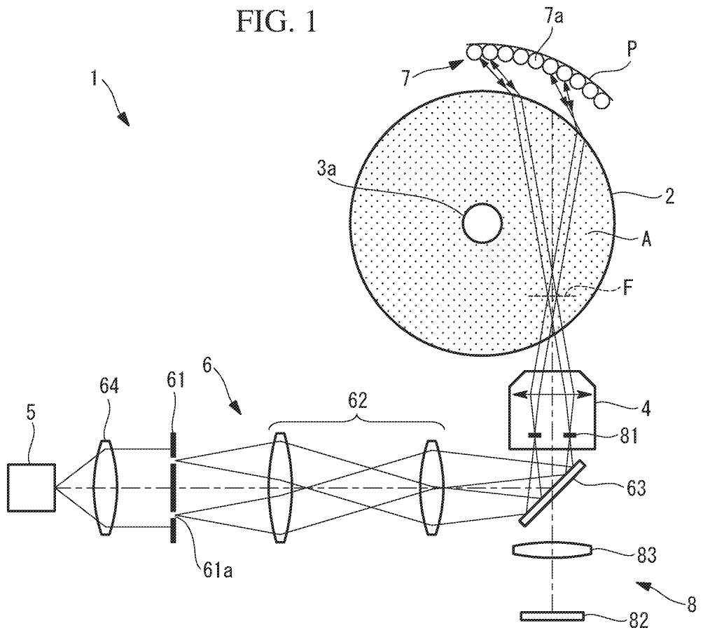

As shown in FIG. 1, the observation apparatus 1 according to this embodiment is used for observing cells cultured in a suspension-culture container 2 from outside the container 2. FIG. 1 illustrates the observation apparatus 1, as viewed from above in the vertical direction.

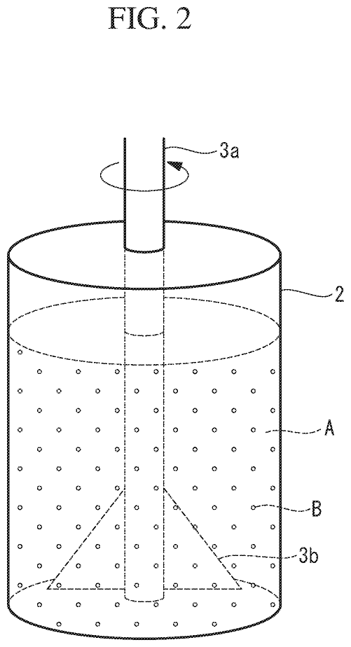

As shown in FIG. 2, the container 2 is of any type composed of a material that is optically transparent with respect to illumination light. The container 2 contains a culture solution A and cells B floating in the culture solution A. The material, shape, and size of the container 2 are not particularly limited. In detail, the container 2 may be composed of a rigid material or a flexible material. The container 2 may have any shape, such as a box shape, a tubular shape, or a bag shape. The size of the container 2 may be large or small. In the drawings, a cylindrical bag composed of a flexible material is illustrated as an example of the container 2. In the container 2, a shaft 3a and stirring blades 3b of a stirrer are disposed. The stirring blades 3b in the culture solution A rotate in accordance with rotation of the shaft 3a, and the rotating stirring blades 3b stir the culture solution A, so that the cells B continue to float in the culture solution A.

The observation apparatus 1 includes an objective lens 4 disposed beside the container 2, an illumination optical system 6 that radiates illumination light from a light source 5 into the container 2 from outside the container 2 via the objective lens 4, a retroreflective member 7 that reflects the illumination light transmitted through the container 2 toward the container 2, and a detection optical system 8 that detects the illumination light collected by the objective lens 4.

The light source 5 is normally used for acquiring a phase-contrast image and is, for example, a lamp light source, such as a mercury lamp, a halogen lamp, a xenon lamp, or a light emitting diode (LED).

The optical axis of the objective lens 4 is disposed substantially in the horizontal direction, and the objective lens 4 is oriented toward the container 2. A focal plane F of the objective lens 4 is disposed inside the container 2.

The illumination optical system 6 includes an aperture stop 61 having a ring-shaped aperture (ring slit) 61a, a relay optical system 62, and a half mirror 63. Reference sign 64 denotes a lens that converts the illumination light output from the light source 5 into collimated light.

The ring slit 61a of the aperture stop 61 is disposed at a position optically conjugate with the pupil position of the objective lens 4. In the aperture stop 61, the illumination light from the lens 64 passes only through the ring slit 61a.

The relay optical system 62 relays the illumination light from the ring slit 61a. Such a relay optical system 62 is constituted of, for example, a pair of convex lenses.

The half mirror 63 reflects a portion (e.g., 20%) of the illumination light from the relay optical system 62 toward the objective lens 4. The half mirror 63 transmits a portion (e.g., 80%) of the illumination light from the objective lens 4.

The illumination light reflected by the half mirror 63 enters the objective lens 4 along the optical axis of the objective lens 4 and is output toward the container 2 from the objective lens 4. Specifically, the objective lens 4 also functions as a part of the illumination optical system 6. The illumination light from the objective lens 4 is transmitted through the sidewall of the container 2, traverses the interior of the container 2 substantially in the horizontal direction, is transmitted again through the sidewall of the container 2, and is output outside the container 2. The position of the aperture stop 61 is adjustable in a direction orthogonal to the optical axis of the illumination light entering the aperture stop 61. By adjusting the position of the aperture stop 61, the position of the illumination light entering the container 2 from the objective lens 4 can be changed in the direction intersecting the optical axis of the illumination light.

The retroreflective member 7 is disposed across from the objective lens 4 with the container 2 interposed therebetween substantially in the horizontal direction. The retroreflective member 7 has an array of multiple small reflective components 7a arranged along a surface P. The surface P intersects with the optical axis of the illumination light transmitted through the container 2. The reflective components 7a are, for example, prisms or spherical glass beads.

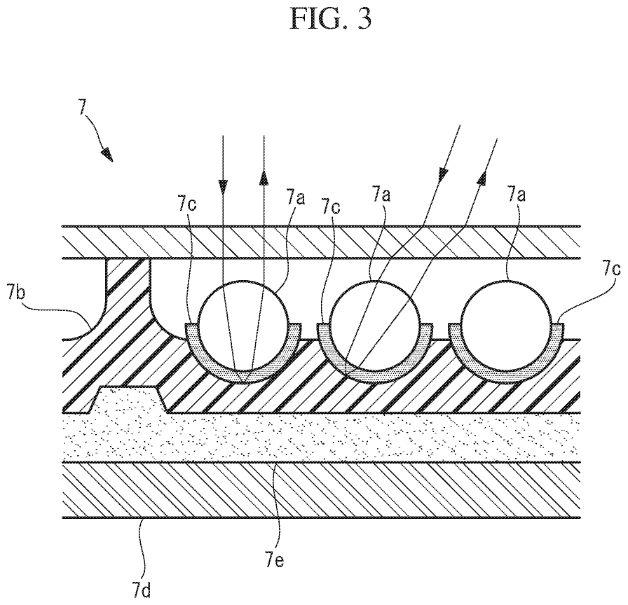

FIG. 3 illustrates an example of the configuration of the retroreflective member 7. As shown in FIG. 3, the multiple reflective components 7a are arranged along the surface of a base member 7b and are each separated from the surface of the base member 7b by a reflective film 7c. In FIG. 3, reference sign 7d denotes a separation film, and reference sign 7e denotes an adhesive for adhering the separation film 7d and the base member 7b to each other.

The illumination light input to the reflective components 7a is reflected by the reflective films 7c and is output from the reflective components 7a in a direction opposite to the input direction. Since the reflective components 7a are small, there is hardly any shifting of the path of the illumination light between the time of input and the time of output. Therefore, the illumination light reflected by the retroreflective member 7 returns along the same path as the path of the illumination light input to the retroreflective member 7. Specifically, the illumination light advances and returns along the same path between the interior of the container 2 and the retroreflective member 7.

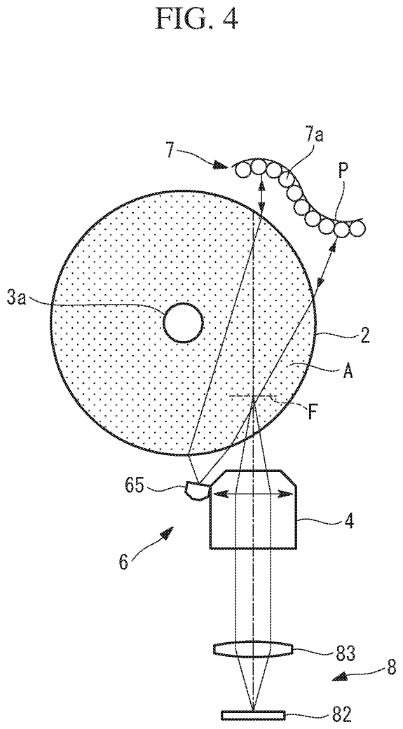

The surface P in which the reflective components 7a are arranged may either be a flat surface or a curved surface. For example, the surface P may be a curved surface having a fixed curvature and curved in one direction, as shown in FIG. 1, or may be a curved surface curved in a plurality of directions, as shown in FIG. 4.

The objective lens 4 and the retroreflective member 7 are disposed at positions where the shaft 3a and the stirring blades 3b of the stirrer do not interfere with the optical path of the illumination light between the objective lens 4 and the retroreflective member 7.

The detection optical system 8 includes a phase film 81 disposed at the pupil position of the objective lens 4, an image capturing element 82, and an imaging lens 83.

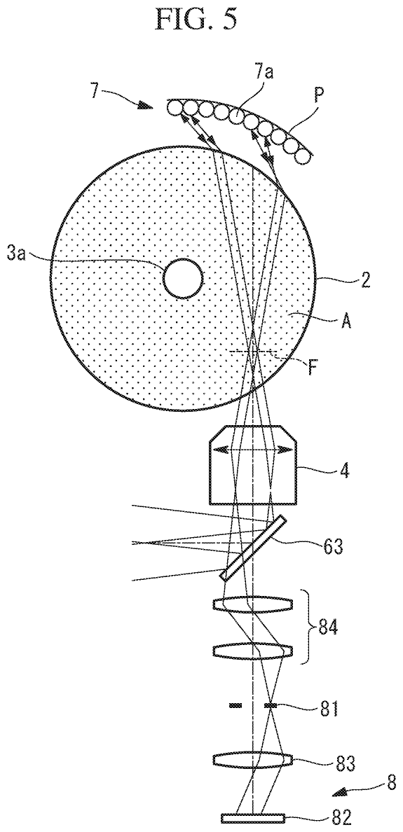

The phase film 81 has a shape corresponding to the shape of the ring slit 61a (i.e., a ring shape). The phase film 81 shifts the phase of the illumination light transmitted through the phase film 81. As shown in FIG. 5, the phase film 81 may be disposed at a position optically conjugate with the pupil position of the objective lens 4. Reference sign 84 denotes a relay optical system that relays the pupil of the objective lens 4 to the phase film 81.

The imaging lens 83 forms an image of the illumination light collected by the objective lens 4 and transmitted through the half mirror 63 onto the image capturing element 82.

The image capturing element 82 is a two-dimensional image sensor (e.g., a CCD image sensor or a CMOS image sensor). The image capturing element 82 captures the image formed by the imaging lens 83 so as to acquire a phase-contrast image of the cells B.

Next, the operation of the observation apparatus 1 will be described.

As shown in FIG. 1, the illumination light from the light source 5 is radiated into the container 2 from the illumination optical system 6 via the objective lens 4. The illumination light is input to the container 2, is transmitted through the culture solution A in the container 2, and is output from the container 2. Then, the illumination light is reflected by the retroreflective member 7, is input again to the container 2, is transmitted through the culture solution A in the container 2 in the opposite direction, and is output from the container 2. Accordingly, the cells B floating in the culture solution A within the container 2 are illuminated in accordance with two types of illumination methods, namely, epi-illumination by the objective lens 4 and trans-illumination by the retroreflective member 7.

While the illumination light is transmitted twice through the container 2, a portion (signal light) of the illumination light is transmitted through the transparent cells B floating in the culture solution A and is refracted. After being transmitted twice through the container 2, the illumination light is input to the objective lens 4, is transmitted through the objective lens 4 and the half mirror 63, and is imaged onto the image capturing element 82 by the imaging lens 83.

In the objective lens 4, the phase film 81 is disposed at a position optically conjugate with the ring slit 61a. The illumination light (refracted light) transmitted through the cells B in the container 2 passes through a position different from the phase film 81 within the objective lens 4, and is output from the objective lens 4. On the other hand, the illumination light (straight-traveling light) not transmitted through the cells B in the container 2 undergoes a phase shift as a result of being transmitted through the phase film 81 within the objective lens 4, and is output from the objective lens 4. Accordingly, an optical image of the cells B formed on the image capturing element 82 has light and dark areas caused by interference between the refracted light and the straight-traveling light, whereby a phase-contrast image of the cells B is acquired by the image capturing element 82.

In this case, as mentioned above, the retroreflective member 7 reflects the illumination light along the same path as that during the input by means of the multiple small reflective components 7a. Therefore, the illumination light input to the container 2 from the retroreflective member 7 is radiated onto the cells B in the container 2 at the same angle from the same direction, regardless of the shape of the sidewall of the container 2 located between the retroreflective member 7 and the interior of the container 2.

For example, in a case where the sidewall of the container 2 is curved or uneven, the sidewall of the container 2 exhibits a lens effect on the illumination light. However, the lens effect is canceled out since the illumination light travels through the sidewall of the container 2 by advancing and returning along the same path. Specifically, the direction and the angle of the illumination light input to the container 2 from the retroreflective member 7 are not affected by the sidewall between the retroreflective member 7 and the interior of the container 2. Therefore, even when the sidewall of the container 2 successively deforms due to the container 2 being composed of a flexible material, or even when the container 2 is replaced with another container 2 having a different shape and a different size, the cells B in the container 2 can be stably illuminated with the illumination light from the retroreflective member 7.

If the sidewall of the container 2 between the objective lens 4 and the interior of the container 2 is flat, the illumination light input to the container 2 from the objective lens 4 travels along the optical axis of the objective lens 4. Specifically, coaxial epi-illumination is realized.

In contrast, if the sidewall of the container 2 between the objective lens 4 and the interior of the container 2 is curved or uneven, the optical axis of the illumination light input to the container 2 from the objective lens 4 tilts relative to the optical axis of the objective lens 4 due to the lens effect of the sidewall of the container 2. As a result, the position of the illumination light (straight-traveling light) returning to the objective lens 4 from the retroreflective member 7 may deviate from the position of the phase film 81 in the direction intersecting the optical axis. In such a case, the aperture stop 61 is positionally adjusted such that the illumination light (straight-traveling light) returning to the objective lens 4 from the retroreflective member 7 is transmitted through the phase film 81, whereby the position of the illumination light to be radiated into the container 2 from the illumination optical system 6 is adjusted.

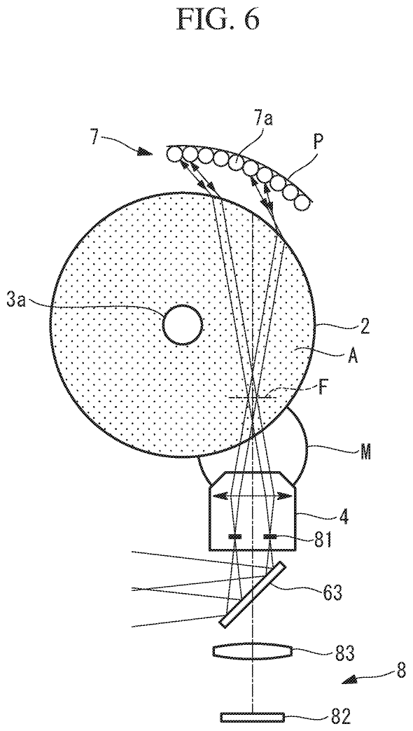

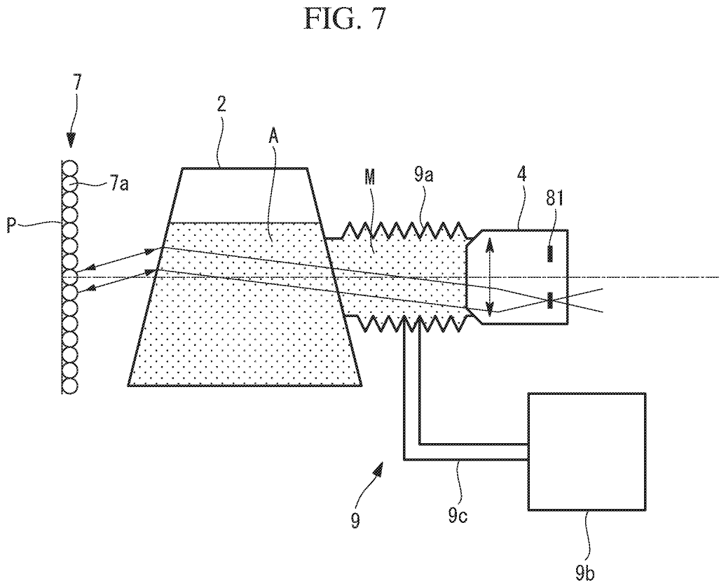

As shown in FIGS. 6 and 7, in this embodiment, a medium M having a refractive index different from that of air may be filled between the objective lens 4 and the container 2. The medium M is, for example, water, oil, gel, or a water absorbing polymer. The refractive index of the medium M is preferably the same as or close to the refractive index of the culture solution A. The refractive index of the medium M may be the same as or close to the refractive index of the material of the container 2.

The lens effect that the sidewall of the container 2 has on the illumination light input to the container 2 from the objective lens 4 is reduced by the medium M between the objective lens 4 and the container 2. Accordingly, if the sidewall of the container 2 is curved or uneven, the direction and the angle of the illumination light to be radiated onto the cells B from the objective lens 4 can be made stable.

As shown in FIG. 6, the medium M may be retained between the objective lens 4 and the container 2 by surface tension of the medium M. Alternatively, as shown in FIG. 7, a mechanism 9 that retains the medium M between the objective lens 4 and the container 2 may be provided.

The mechanism 9 includes, for example, a tubular wall 9a that seals the space between an end surface of the objective lens 4 and the sidewall of the container 2, a container 9b that contains the medium M having fluidity, and a pipe 9c that connects the interior of the wall 9a and the interior of the container 9b. The medium M is supplied from the container 9b to the interior of the wall 9a via the pipe 9c. The wall 9a is preferably expandable and contractible in the longitudinal direction (i.e., the direction parallel to the optical axis of the objective lens 4). For example, the wall 9a may have a bellows structure. By expanding and contracting the wall 9a, the objective lens 4 can be moved along the optical axis while the sealability inside the wall 9a is maintained.

As an alternative to this embodiment in which the illumination optical system 6 radiates the illumination light into the container 2 via the objective lens 4, the illumination light may be radiated into the container 2 without the intervention of the objective lens 4, as shown in FIG. 4. The illumination optical system 6 in FIG. 4 includes a light source 65 that is disposed beside the objective lens 4 and that emits illumination light. In order to dispose the optical axis of the illumination light as close to the optical axis of the objective lens 4 as possible, the light source 65 is preferably disposed near the objective lens 4.

As an alternative to this embodiment in which the illumination light is radiated into the container 2 from the illumination optical system 6 substantially in the horizontal direction, the illumination light may be radiated into the container 2 from the illumination optical system 6 in a direction other than the horizontal direction.

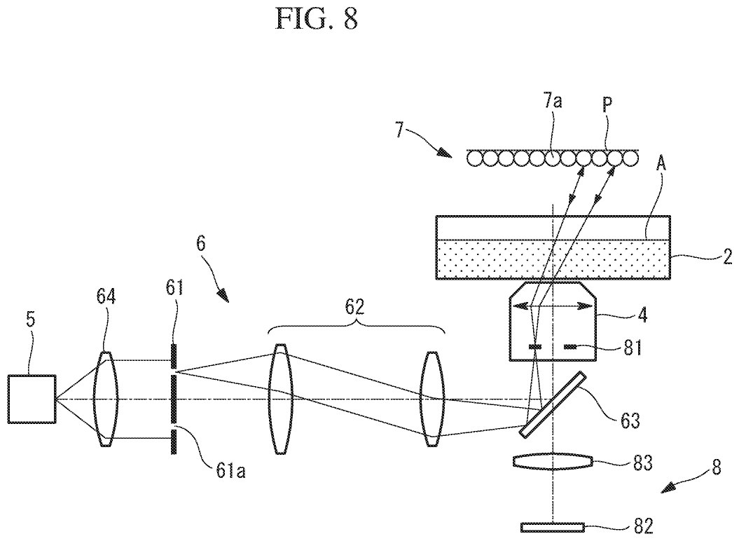

For example, as shown in FIG. 8, the illumination light may be radiated upward into the container 2 from the illumination optical system 6. In the modification in FIG. 8, the objective lens 4 is disposed below the container 2, and the retroreflective member 7 is disposed above the container 2.

The liquid surface of the culture solution A is concave due to surface tension, and exhibits a lens effect on the illumination light. According to the modification in FIG. 8, the lens effect that the liquid surface of the culture solution A has on the illumination light can be canceled out by using the retroreflective member 7.

As an alternative to this embodiment in which a phase-contrast image of the cells B is observed by using the ring slit 61a and the phase film 81, a bright-field image of the cells B may be observed. Specifically, as shown in FIG. 4 or 5, the illumination optical system 6 does not have to be provided with the aperture stop 61, and the detection optical system 8 does not have to be provided with the phase film 81. In the modifications of FIGS. 4 and 5, an episcopic bright-field image and a transmission bright-field image of the cells B are observed.

A configuration similar to that in this embodiment may be applied to episcopic differential-interference observation.

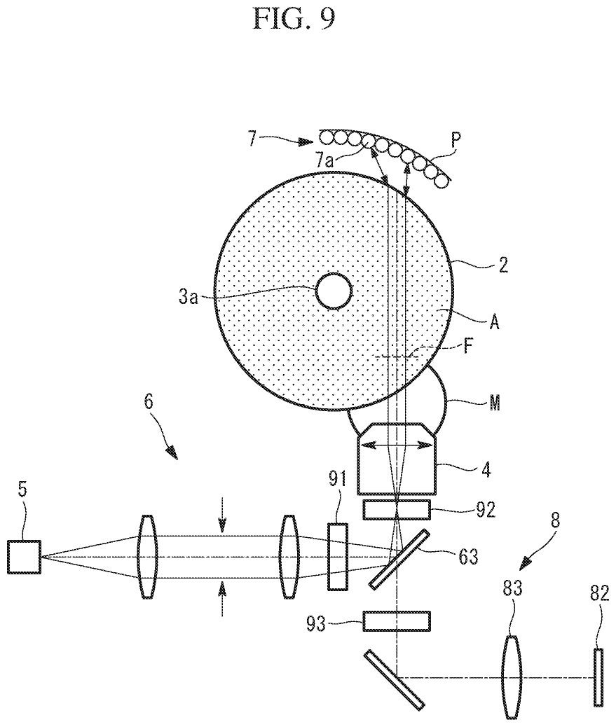

In this case, as shown in FIG. 9, the illumination optical system 6 may include a polarizer 91 that allows the illumination light from the light source 5 to pass therethrough, the observation apparatus 1 may include a birefringent element 92 near the pupil position of the objective lens 4, and the detection optical system 8 may include an analyzer (crossed-Nicol) 93. The birefringent element 92 is, for example, a differential-interference-contrast (DIC) prism that transmits illumination light transmitted through the polarizer 91 and that also transmits signal light collected by the objective lens 4 from the cells B. The analyzer 93 transmits the signal light transmitted through the birefringent element 92 from the cells B.

The illumination light from the light source 5 is transmitted through the polarizer 91 so that the polarization direction thereof is set to one direction, and is transmitted through the birefringent element 92 so as to be split into two beams of illumination light with different polarization directions. Subsequently, the two beams of illumination light are transmitted through the cells B. The two beams of illumination light with different optical paths are given an optical path difference as they are transmitted through the cells B having various thicknesses. The two beams of illumination light are reflected by the retroreflective member 7 and are subsequently given an optical path difference by being transmitted again through the same positions of the cells B.

Then, the two beams of illumination light are combined on the same optical path by passing through the birefringent element 92 again, and pass through the analyzer 93. Accordingly, a contrast occurs between light and dark areas due to interference between the two beams of illumination light having the optical path difference, so that the cells B can be observed in a differential interference image.

Even in this case, the beams of illumination light transmitted through the positions of the cells B are caused to pass through the same positions again by the retroreflective member 7, so that a phase contrast occurring due to birefringence can be doubled.

A configuration similar to that in this embodiment may be applied to transmission observation based on oblique illumination.

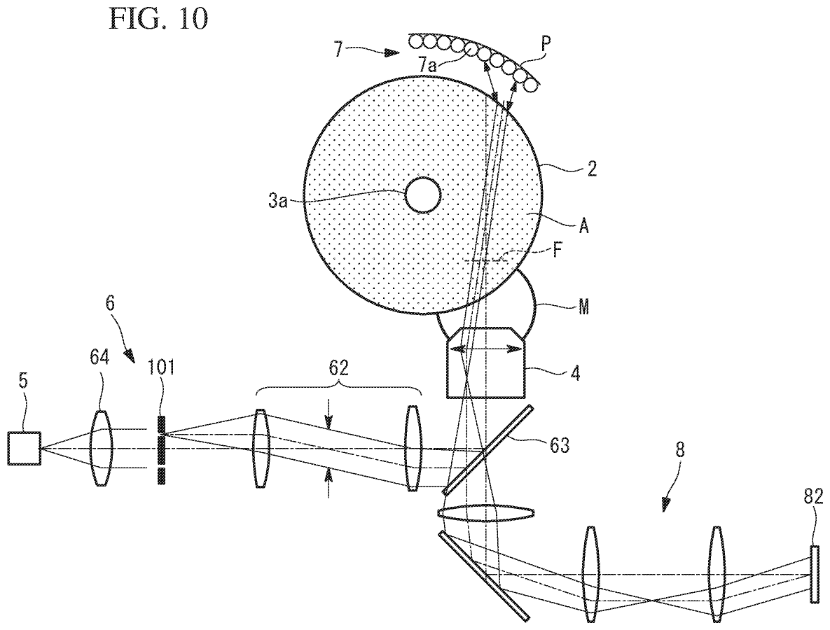

In this case, as shown in FIG. 10, the illumination optical system 6 may have a ring slit (aperture) 101 disposed at a position optically conjugate with the pupil position of the objective lens 4 and away from the center of the optical axis in the radial direction, and may cause illumination light to enter the cells B at a specific angle.

The illumination light transmitted through the cells B in an oblique direction relative to the optical axis of the objective lens 4 is reflected by the retroreflective member 7, thereby generating oblique illumination in which the illumination light is radiated onto the cells Bb obliquely relative to the optical axis of the objective lens 4 from the opposite side of the objective lens 4. Then, the illumination light transmitted through the cells B is split off by the half mirror 63 and is captured by the image capturing element 82, such as a CCD, so that a three-dimensional image of the cells B can be observed.

A configuration similar to that of the observation apparatus in FIG. 10 may be applied to dark-field observation.

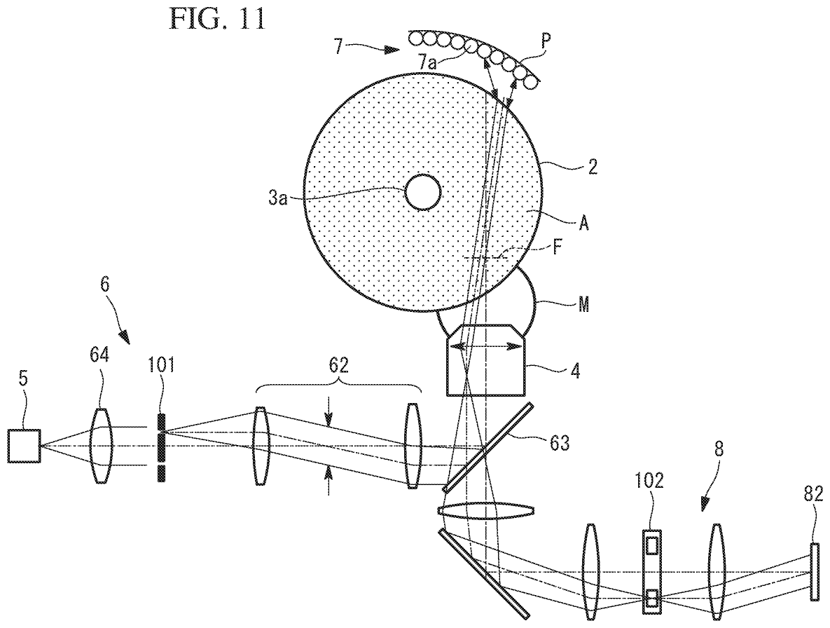

In this case, as shown in FIG. 11, the detection optical system 8 may include a light attenuating member 102 disposed at a position that is located near a position optically conjugate with the pupil position of the objective lens 4 and that corresponds to the ring slit (aperture) 101. The light attenuating member 102 is, for example, an aperture stop that blocks a portion of the illumination light, or a neutral density (ND) filter.

According to this configuration, the amount of direct light transmitted as oblique illumination light through the cells B from the retroreflective member 7 can be reduced by the light attenuating member 102, whereby dark-field observation can be performed.

As an alternative to the example applied to dark-field observation shown in FIG. 11, this configuration is advantageous in that phase-contrast observation can be performed without using a dedicated phase-contrast observation objective lens by having a phase film in place of the light attenuating member 102.

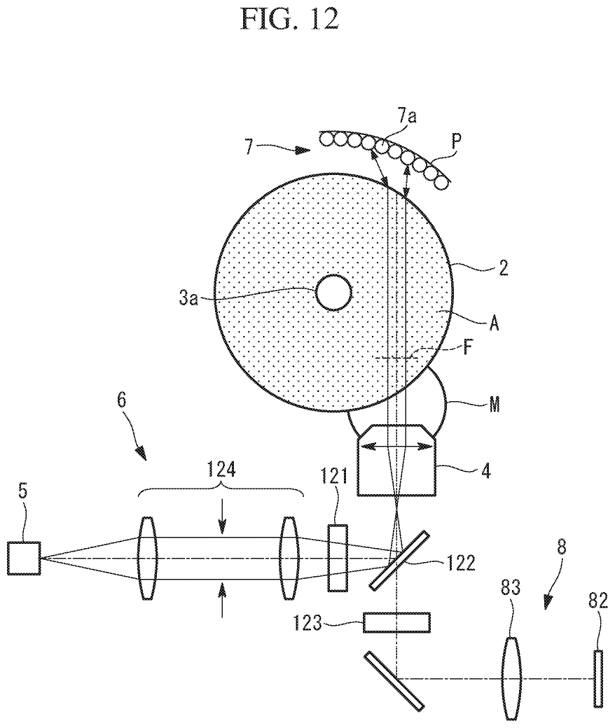

In this embodiment, a configuration that allows for fluorescence observation is also possible, as shown in FIG. 12.

In this case, the illumination optical system 6 includes an excitation filter 121, and the detection optical system 8 includes a dichroic mirror 122 and an excitation-light cut filter 123. The illumination light output from the light source 5 is relayed by a relay optical system 124 and is turned into excitation light by being transmitted through the excitation filter 121. The excitation light is then radiated onto the cells B. A fluorescent material contained in the cells B is excited as a result of being irradiated with the excitation light, so that fluorescence (signal light) is generated from the cells B. The fluorescence is split off from the optical path of the illumination optical system 6 by the dichroic mirror 122 and is captured by the image capturing element 82 after the excitation light is removed from the fluorescence by the excitation-light cut filter 123. Accordingly, fluorescence observation can be performed.

A configuration similar to that of the observation apparatus in FIG. 12 may be applied to laser-scanning confocal epi-fluorescence observation.

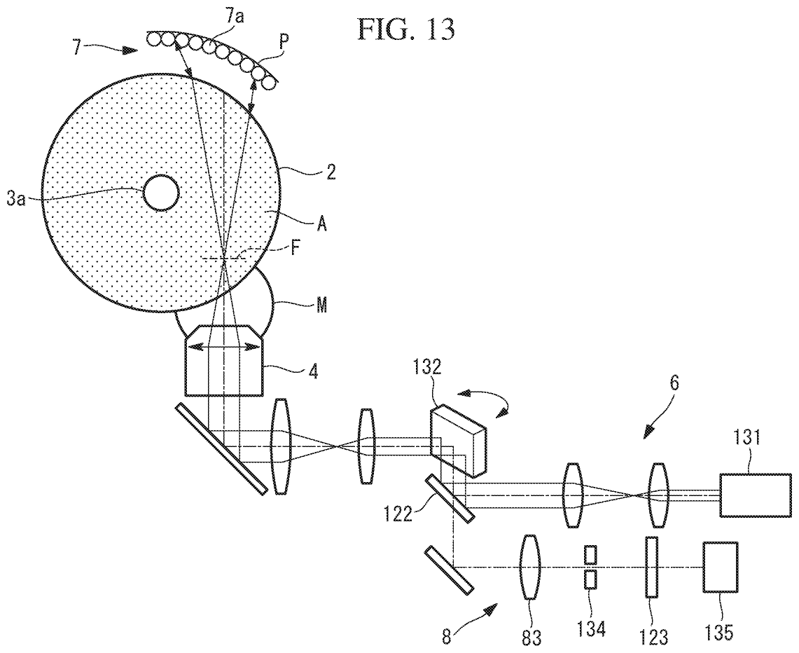

In this case, as shown in FIG. 13, the illumination optical system 6 includes a laser light source 131 and a scanner 132, and the detection optical system 8 includes a confocal pin hole 134 and a light detector 135. The light detector 135 is, for example, a photomultiplier tube (PMT).

Laser light (excitation light) from the laser light source 131 is input to the container 2 by the illumination optical system 6 and the objective lens 4 and is focused onto the focal plane F of the objective lens 4, so that a light spot is formed. The light spot is two-dimensionally scanned by the scanner 132.

If there are cells B in the scan range for the light spot, fluorescence is generated at each scan position of the light spot as a result of the fluorescent material contained in the cells B being excited, and the generated fluorescence is output in all directions from each scan position. A portion of the fluorescence generated from each scan position is transmitted through the container 2, is focused by the objective lens 4, and is split off from the optical path of the laser light by the dichroic mirror 122 in the course of returning along the optical path of the laser light via the scanner 132. Subsequently, the fluorescence passes through the imaging lens 83, the confocal pin hole 134, and the excitation-light cut filter 123 and is detected by the light detector 135.

Because the cells B are transparent, a portion of the laser light input to the cells B is transmitted through the cells B and is output from the container 2 toward the opposite side from the objective lens 4. The output laser light is reflected by the retroreflective member 7 and travels along the same path so as to enter the cells B again from the opposite side from the objective lens 4.

In this case, the retroreflective member 7 reflects the laser light by means of the multiple small reflective components 7a such that the laser light returns along the same path with hardly any shifting of the path. Accordingly, a light spot of the laser light can be formed again at substantially the same position as the first scan position, regardless of the state of, for example, curvature of the container 2.

Specifically, since the laser light is radiated twice onto the same scan position, the fluorescence generated at each scan position can be substantially doubled. This is advantageous in that a bright fluorescence image can be acquired.

With regard to the fluorescence generated in the entire region of the container 2 through which the laser light passes, fluorescence generated in regions other than the light spot formed at the focal position of the objective lens 4 cannot pass through the confocal pin hole 134 and thus cannot be detected by the light detector 135.

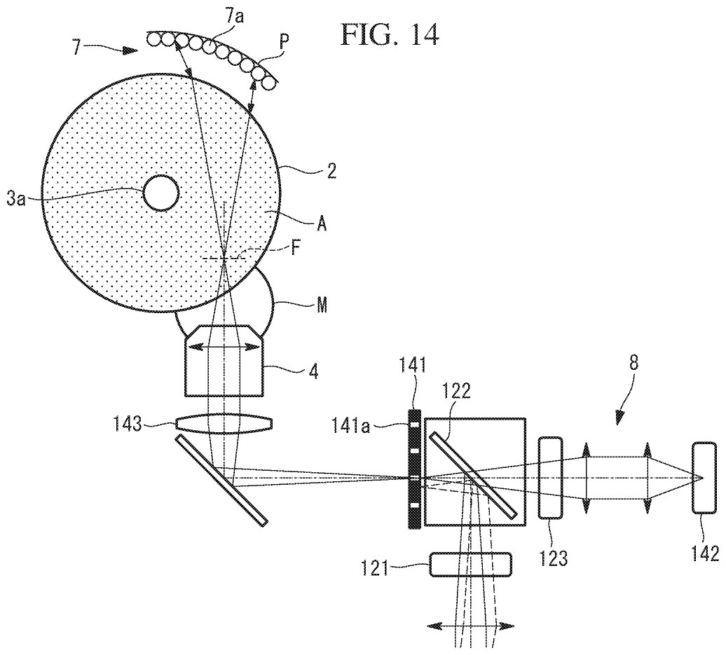

In FIG. 13, the laser scanning type shown as an example of a confocal fluorescence observation type includes the scanner 132 and the confocal pin hole 134. Alternatively, a type that includes a confocal disk 141 may be employed, as shown in FIG. 14.

The confocal disk 141 is disposed at a position optically conjugate with the focal position of the objective lens 4 and has a plurality of pin holes 141a through which excitation light and fluorescence can pass. The detection optical system 8 includes an image capturing element 142, such as a CCD image sensor, capable of simultaneously detecting the fluorescence passing through the plurality of pin holes 141a.

Excitation light is generated by the excitation filter 121 from the illumination light from the light source 5. The generated excitation light passes through the confocal disk 141 and is focused by a focusing lens 143. Accordingly, multiple light spots are formed at the focal position, disposed in the container 2, of the objective lens 4. The multiple light spots can be scanned in the container 2 by, for example, rotating the confocal disk 141.

The fluorescence generated at each scan position passes through the pin holes 141a in the confocal disk 141, is subsequently split off from the optical path of the excitation light by the dichroic mirror 122, and is captured by the image capturing element 142 after the excitation light is removed by the excitation-light cut filter 123.

In this case, the excitation light is radiated twice onto the position of each light spot by the retroreflective member 7. The fluorescence generated at the position of each light spot is reflected by the retroreflective member 7, so as to be detected as a portion of the fluorescence generated from the light spot. Accordingly, this is advantageous in that a bright fluorescence image can be acquired.

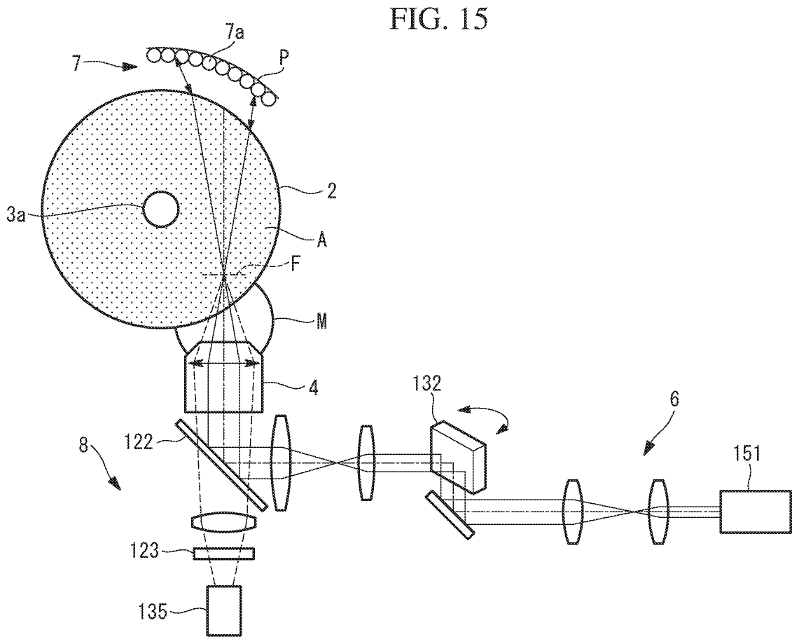

As shown in FIG. 15, in fluorescence observation, a light source 151 that outputs extremely-short pulse laser light for multiphoton fluorescence observation may be used in place of the light source 5.

The observation apparatus in FIG. 15 is different from the fluorescence observation apparatus described above in that the dichroic mirror 122 of the detection optical system 8 is disposed near the objective lens 4, and that the confocal pin holes 134 and 141 have been removed.

The extremely-short pulse laser light from the light source 151 is scanned by the scanner 132 and is focused onto the focal position of the objective lens 4, so that a light spot is formed. At the light spot at the focal position, the photon density increases. Consequently, fluorescence is generated limitedly at the position of the light spot in accordance with a multiphoton excitation effect. Of the generated fluorescence, fluorescence output toward the objective lens 4 is focused by the objective lens 4, is split off from the optical path of the extremely-short pulse laser light by the dichroic mirror 122, has the laser light component removed therefrom by the excitation-light cut filter 123, and is detected by the light detector 135. Accordingly, a fluorescence image can be acquired.

Similar to the laser-scanning confocal fluorescence observation, the extremely-short pulse laser light is reflected by the retroreflective member 7. However, since the extremely-short pulse laser light is reflected with the wave front thereof being split at the position of the light spot in the container 2 that has received the light again, the pulse width increases so that the multiphoton excitation effect does not occur. Therefore, unlike the laser-scanning confocal fluorescence observation, the increasing effect of the amount of fluorescence caused by radiating the excitation light twice is not achieved. However, since the fluorescence is generated limitedly at the position of the light spot, flare does not occur even if a small shift occurs due to the reflective components 7a. Thus, the fluorescence output toward the retroreflective member 7 is returned to the same position in the container 2 by the retroreflective member 7, and can be focused by the objective lens 4. Accordingly, fluorescence that is discarded in normal episcopic observation is captured. This is advantageous in that a bright fluorescence image can be acquired.

A configuration similar to that in FIG. 15 may employ an observation apparatus that detects secondary harmonic generation (SHG) and tertiary harmonic generation (THG) induced in the cells B as a result of inputting extremely-short pulse laser light in place of fluorescence generated by the multiphoton excitation effect.

In this case, the light source 151 used outputs, for example, extremely-short pulse laser light with a wavelength of 1200 nm. The excitation-light cut filter 123 used blocks extremely-short pulse laser light with a wavelength of 1200 nm and transmits extremely-short pulse laser light with wavelengths of 600 nm and 400 nm.

By detecting a harmonic (signal light) generated in accordance with a nonlinear effect by a specific substance in the cells B, transparent cells can be detected without having to fluorescently label the cells. Normally, a large amount of harmonic occurs in the transmission direction opposite from the input direction of the extremely-short pulse laser light. According to this example, a harmonic occurring from the cells B at the opposite side from the objective lens 4 is returned toward the cells B by the retroreflective member 7. Accordingly, this is advantageous in that a harmonic can be efficiently detected by a compact episcopic configuration.

In the fluorescence observation described above, an optical filter that is disposed near the retroreflective member 7 and that blocks fluorescence may be provided.

Of the fluorescence generated in the cells B as result of the cells B being irradiated with laser light, the optical filter blocks fluorescence output toward the retroreflective member 7. Accordingly, for example, the optical filter is disposed between the container 2 and the retroreflective member 7.

In a case where the cells B have strong scattering properties, the fluorescence generated in the cells B is output toward the retroreflective member 7. The fluorescence reflected by the retroreflective member 7 may decrease the contrast by being scattered again by the cells B. By disposing the optical filter between the retroreflective member 7 and the cells B, the fluorescence output toward the retroreflective member 7 is blocked by the optical filter, so that the excitation light alone is transmitted through the optical filter. Then, the excitation light alone is reflected by the retroreflective member 7 and is input again to the cells B. Accordingly, the fluorescence intensity can be doubled while a decrease in the contrast can be prevented.

In detail, the excitation light transmitted through the individual positions of the cells B is reflected by the retroreflective member 7 and is input again to the same positions of the cells B, so that the fluorescence generated at the individual positions of the cells B can be substantially doubled.

Accordingly, a bright fluorescence image can be acquired.

In this case, if the light source is not a point light source (e.g., a mercury light source), off-axis excitation light is radiated onto the cells B, in addition to on-axis excitation light. In the observation apparatus according to this embodiment, both on-axis excitation light and off-axis excitation light are reflected by the retroreflective member 7 so as to return along the same path, so that the aforementioned advantages can be achieved.

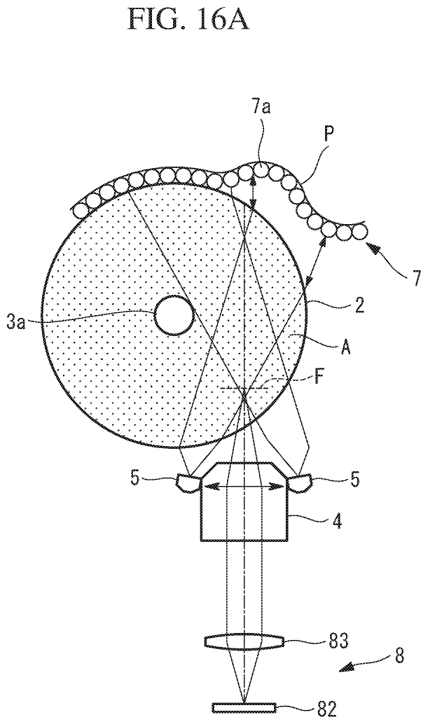

In the above-described embodiment and modifications, the light source 5 may be disposed around the objective lens 4, as shown in FIG. 16A. In particular, the light source 5 may be ring-shaped, as shown in FIG. 16B.

With this arrangement, scattered light scattered by the cells B can be observed, so that observation based on oblique illumination can be performed. In the case of fluorescence observation, excitation light is radiated onto the cells B from outside the optical axis of the detection optical system 8, so that the amount of excitation light focused by the objective lens 4 is reduced, whereby a favorable fluorescence image can be acquired.

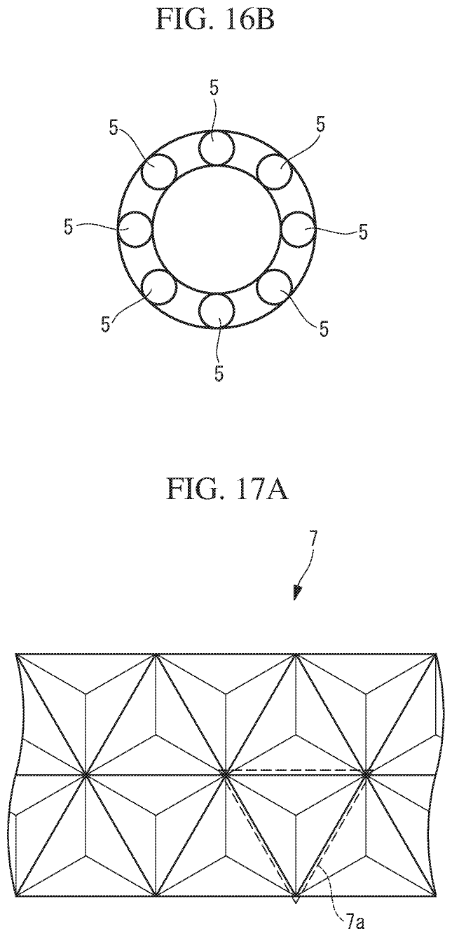

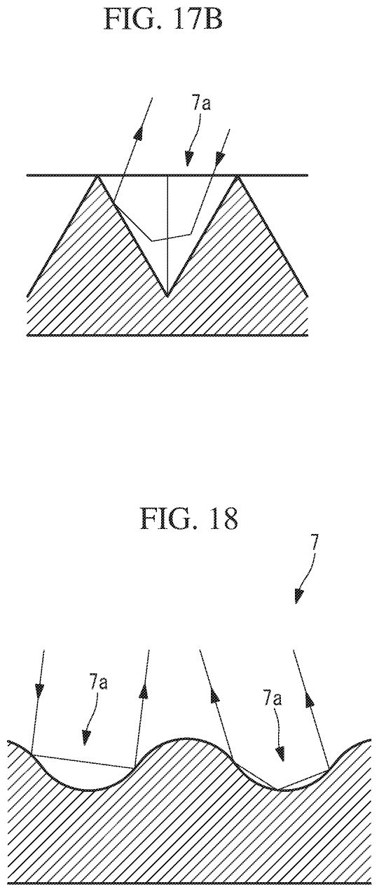

FIGS. 17A to 18 illustrate modifications of the structure of the retroreflective member 7. In the above-described embodiment and modifications, the retroreflective member 7 used may have protrusions and recesses, as shown in FIGS. 17A to 18.

As shown in FIGS. 17A and 17B, the retroreflective member 7 may be a reflective member having a geometrical shape and may be configured such that reflected illumination light returns along the same path as the path of illumination light input to the retroreflective member 7. In the examples in FIGS. 17A and 17B, each reflective component 7a is constituted of three reflective surfaces.

Alternatively, as shown in FIG. 18, the retroreflective member 7 may be a reflective member having a wavy surface and may be configured such that reflected illumination light returns along the same path as the path of illumination light input to the retroreflective member 7.

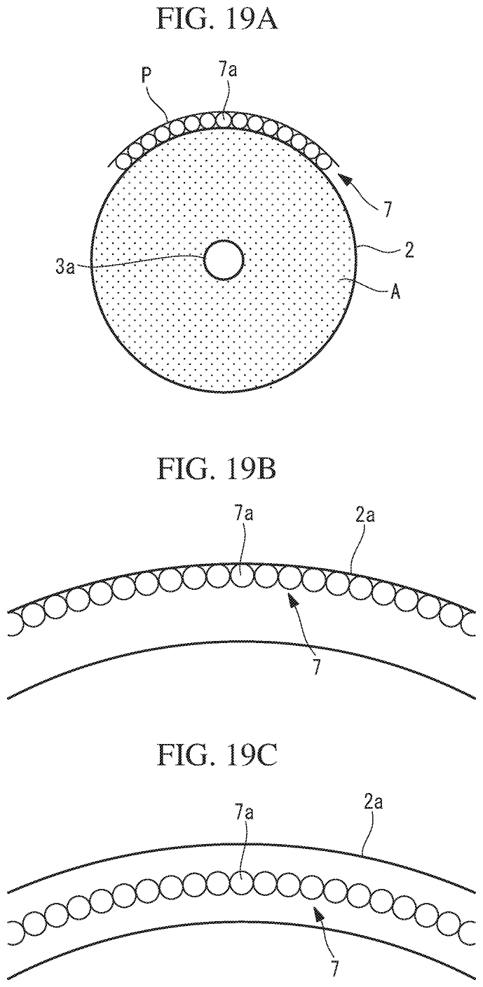

In the above-described embodiment and modifications, the retroreflective member 7 is disposed outside the container 2. Alternatively, as shown in FIG. 19A, the retroreflective member 7 may be integrated with the container 2.

The retroreflective member 7 may be provided at any position of the container 2 so long as the retroreflective member 7 can exhibit its function. For example, as shown in FIG. 19B, the retroreflective member 7 may be provided along the outer surface of a wall 2a of the container 2. Alternatively, as shown in FIG. 19C, the retroreflective member 7 may be provided inside the wall 2a of the container 2.

The container 2 used in the above-described embodiment and modifications is preferably composed of an optically transparent material. The material of the container 2 preferably has a refractive index Nd ranging between 1.3 and 2. For example, the material of the container 2 is preferably fluoroplastic or glass.

Although an observation apparatus has been described above, the present invention also includes an observation method for observing cells floating in a container by using a retroreflective member.

An example of an observation method for observing cells floating in a container includes:

(A) a radiating step of radiating illumination light onto the cells in the container;

(B) a reflecting step of retroreflecting light transmitted through the cells; and

(C) a capturing step of capturing an image of the light retroreflected in the reflecting step and transmitted through or scattered by the cells.

Another example of an observation method for observing cells floating in a container includes:

(a) a radiating step of radiating illumination light onto the cells in the container;

(b) a reflecting step of retroreflecting the light radiated onto the cells in the radiating step and transmitted through the cells; and

(c) a capturing step of capturing an image of fluorescence generated from the cells in accordance with the illumination light radiated onto the cells in the radiating step and/or the retroreflecting step.

The retroreflection described in the step (C) or (c) implies that the input angle and output angle of the illumination light are the same or substantially the same, which is realized by the aforementioned small reflective components.

As a result, the following aspect is read from the above-described embodiment of the present invention.

An aspect of the present invention provides an observation apparatus for observing a cell in a suspension-culture container. The observation apparatus includes: an illumination optical system that radiates illumination light into the container from outside the container; an objective lens that collects signal light from the cell in the container; a detection optical system that detects the signal light collected by the objective lens; and a retroreflective member that has an array of a plurality of small reflective components, is disposed across from the illumination optical system with the container interposed therebetween, and reflects the illumination light transmitted through the container.

According to this aspect, the illumination light output from the illumination optical system is transmitted through the interior of the container. Subsequently, the illumination light reflected by the retroreflective member is transmitted again through the interior of the container. Specifically, the cell in the container is irradiated with the illumination light twice from opposite sides of the container. In the container, the signal light from the cell is generated in accordance with the irradiation of the illumination light. The signal light output outside the container is collected by the objective lens and is detected by the detection optical system. Accordingly, the cell inside the container can be observed.

In this case, the array of small reflective components of the retroreflective member reflects the illumination light along the same path as the input illumination light. Specifically, the illumination light is transmitted twice through a wall of the container, located between the retroreflective member and the interior of the container, in opposite directions along the same path, so that a lens effect that the wall of the container between the retroreflective member and the interior of the container has on the illumination light is canceled out. Thus, the illumination light input to the container from the retroreflective member is not affected by the wall of the container between the retroreflective member and the interior of the container. Accordingly, the cell in the container can be stably illuminated with the illumination light, regardless of the type of the container.

In the above aspect, the illumination optical system may radiate the illumination light into the container via the objective lens.

According to this configuration, observation of reflected light or scattered light from the cell by using coaxial epi-illumination and observation of transmitted light from the cell by using trans-illumination can both be achieved. Specifically, the illumination light that has traveled through the objective lens is radiated onto the cell along or substantially along the optical axis of the objective lens, and the illumination light (signal light) reflected or scattered by the cell is collected by the objective lens. Accordingly, an episcopic bright-field image of the cell can be observed. On the other hand, the illumination light reflected by the retroreflective member is radiated onto the cell along or substantially along the optical axis of the objective lens, and the illumination light (signal light) transmitted through the cell is collected by the objective lens. Accordingly, a transmission bright-field image of the cell can be observed.

In the optical path to the retroreflective member, the occurrence of vignetting of the illumination light can be reduced.

In the above aspect, the illumination optical system may have an aperture disposed at a position optically conjugate with a pupil position of the objective lens, and the detection optical system may include a phase film that is disposed at the pupil position of the objective lens or at a position optically conjugate with the pupil position and that has a shape corresponding to a shape of the aperture.

According to this configuration, phase-contrast observation using coaxial epi-illumination can be performed. Specifically, the illumination light passed through the aperture in the illumination optical system is transmitted twice through the interior of the container and is input to the objective lens. While being transmitted through the interior of the container, a portion of the illumination light is diffracted as it passes through the cell, whereas the remaining portion of the illumination light travels straight without being diffracted. The non-diffracted straight-traveling light passes through the phase film disposed at the position optically conjugate with the aperture, so that a phase shift occurs. Then, the straight-traveling light and the diffracted light interfere with each other, so that the cell, which is transparent, can be observed in accordance with bright and dark areas.

In the above aspect, the observation apparatus may further include a mechanism that retains a medium having a refractive index different from a refractive index of air between the objective lens and the container.

In a case where the wall of the container at the position where the illumination light is transmitted is curved or uneven, the wall of the container exhibits a lens effect on the illumination light. The medium retained between the objective lens and the wall of the container reduces the lens effect of the wall of the container, so that the cell in the container can be illuminated more stably.

### REFERENCE SIGNS LIST

1 observation apparatus
2 container
3a shaft
3b stirring blade
4 objective lens
5 light source
6 illumination optical system
61 aperture stop
61a ring slit, aperture
62 relay optical system
63 half mirror
7 retroreflective member
7a reflective component
8 detection optical system
81 phase film
82 image capturing element
83 imaging lens
9 mechanism
A culture solution
B cell
M medium

1. An observation apparatus for observing a cell in a suspension-culture container, the observation apparatus comprising:
an illumination optical system that is configured to radiate illumination light into the container from outside the container;
an objective lens that is configured to collect signal light from the cell in the container;
a detection optical system that is configured to detect the signal light collected by the objective lens; and
a retroreflective member that has an array of a plurality of small reflective components, is disposed across from the illumination optical system with the container interposed therebetween, and is configured to reflect the illumination light transmitted through the container.

2. The observation apparatus according to claim 1,
wherein the illumination optical system radiates the illumination light into the container via the objective lens.

3. The observation apparatus according to claim 2,
wherein the illumination optical system has an aperture disposed at a position optically conjugate with a pupil position of the objective lens, and
wherein the detection optical system includes a phase film that is disposed at the pupil position of the objective lens or at a position optically conjugate with the pupil position and that has a shape corresponding to a shape of the aperture.

4. The observation apparatus according to claim 1, further comprising:
a mechanism that is configured to retain a medium, having a refractive index different from a refractive index of air, between the objective lens and the container.

5. The observation apparatus according to claim 1,
wherein each of the reflective components has a prism or a glass bead.

6. The observation apparatus according to claim 1,
wherein the container has a bioreactor or a bag.

7. The observation apparatus according to claim 1,
wherein a stirring blade is disposed inside the container.

8. The observation apparatus according to claim 1,
wherein the signal light is any one of transmitted light transmitted through the cell, scattered light scattered by the cell, and fluorescence generated from the cell.

9. The observation apparatus according to claim 1,
wherein the cell is a floating cell floating in the container.

10. An observation method for observing a cell floating in a container, the observation method comprising:
a radiating step of radiating illumination light onto the cell floating in the container;
a reflecting step of retroreflecting the illumination light radiated onto the cell in the radiating step and transmitted through the cell; and
a capturing step of capturing an image of transmitted light obtained as a result of the illumination light retroreflected in the reflecting step being transmitted again through the cell or an image of scattered light obtained as a result of the illumination light retroreflected in the reflecting step being scattered by the cell.

11. An observation method for observing a cell floating in a container, the observation method comprising:
a radiating step of radiating illumination light onto the cell floating in the container;
a reflecting step of retroreflecting the illumination light radiated onto the cell in the radiating step and transmitted through the cell; and
a capturing step of capturing an image of fluorescence generated from the cell in accordance with the illumination light radiated onto the cell in the radiating step and/or the reflecting step.
